(19) United States
(12) Patent Application Publication
(10) Pub. No.: US 2018/0348213 A1
(43) Pub. Date: Dec. 6, 2018

(54) CENTRIFUGE-FREE ISOLATION AND DETECTION OF RARE CELLS

(71) Applicant: Purdue Research Foundation, West Lafayette, IN (US)

(72) Inventors: Cagri A. Savran, West Lafayette, IN (US); Chun-Li Chang, West Lafayette, IN (US); Wanfeng Huang, San Jose, CA (US)

(21) Appl. No.: 15/761,701

(22) PCT Filed: Sep. 22, 2016
(86) PCT No.: PCT/US16/53201

§ 371 Date: Mar. 20, 2018

Related U.S. Application Data
(60) Provisional application No. 62/222,193, filed on Sep. 22, 2015.

(51) Int. Cl. G01N 33/543; G01N 1/38; G01N 1/40; G01N 33/569

(52) U.S. Cl. CPC G01N 33/54333; G01N 33/56966; G01N 1/4077; G01N 1/38

(57) ABSTRACT

Additive techniques, including a direct-dilution method and a direct-incubation method, are described for isolating target entities in a fluid sample. In some implementations, a volume of a diluent is added to a fluid sample to generate a first mixture. The volume of the diluent added may be sufficient to obtain a specified viscosity of the first mixture lower than a viscosity of the fluid sample. A number of binding moiety-conjugated magnetic beads are added to the first mixture to generate a second mixture. The second mixture is incubated for a time that is sufficient for the binding moiety-conjugated magnetic beads to bind to rare target entities in the second mixture. A portion of the second mixture is injected into a fluidic chamber. A magnetic force is applied to attract the magnetized rare target entities in the second mixture to an isolation surface within the fluidic chamber.

### CROSS-REFERENCE TO RELATED APPLICATIONS

This application claims the benefit of U.S. Provisional Patent Application No. 62/222,193 filed on Sep. 22, 2015 and entitled “METHOD FOR CENTRIFUGE-FREE ISOLATION AND DETECTION OF CELLS FROM WHOLE BLOOD,” which is incorporated herein by reference in its entirety.

### TECHNICAL FIELD

The present application relates to methods of isolating target particles, such as cells, in a biological fluid sample.

### BACKGROUND

The isolation, detection, and/or capture of target entities, such as cells, present in a fluid sample, such as bodily fluids, e.g., whole blood, is highly significant, because the captured cells may be an indication of a pathological condition or a disease. The cells can be enumerated for correlation with the disease state, subjected to genetic analysis or cultured and used to test combinations of drugs or to discover new drugs. Specially, the isolation and detection of rare cells in bodily fluids such as blood is of particular importance, but is difficult, because of the very low numbers of such rare cells in fluid samples.

Circulating tumor cells (or CTCs) in a patient's blood and fetal cells in maternal blood including fetal nucleated red blood cells, fetal white blood cells, and fetal trophoblasts are examples of such rare cells. The majority of cancer-related deaths are due to metastasis of tumor cells to various other tissue and organ structures that may be distant from the originating tumor. CTCs can detach from primary and metastatic tumors and enter into the vascular system. Early detection of CTCs can play a significant role in improving survival rate.

Detection of CTCs can further be used to ascertain efficacy of treatment, e.g., chemotherapy, radiation, surgery, etc. Presence of CTCs after such treatments may be indicative of recurrence of cancer. CTCs and other rare cells can be indicative of rare events, and hold the key to a plethora of unanswered biological and medical questions. The rare cells can also be subjected to further downstream tests and analysis after detection and enumeration. For example, they can be introduced (e.g. by grafting) in animal to study metastatic models as well as sequenced to interrogate the genome and the transcriptome which could reveal mutations and quantitate gene expressions. In addition, CTCs have the potential to be cultured, grown, and used for understanding the biology of metastasis as well as testing of drugs, paving the way to personalized medicine.

Traditional extraction techniques for CTCs and other rare cells, and even other cells not typically considered to be rare, often include using centrifugation or other subtractive techniques to separate red blood cells (RBCs) from other cells of similar size such as white blood cells (WBCs) and CTCs, and plasma into distinct layers based on the mass associated with each component in whole blood. Such techniques often assume that plasma is mostly devoid of cells, and therefore the plasma is removed to prevent non-specific binding. Plasma is often removed using an aspirator to apply negative pressure to suction the plasma from a sample container that has been centrifuged.

CTCs and other rare cells are often challenging to detect in small volumes of whole blood due to their concentration often being as low as one cell per milliliter of whole blood. Thus, extraction protocols that use centrifugation to remove plasma often require a large volume of whole blood in order to capture and extract a sufficient number CTCs to be analyzed and/or harvested. A reliable analysis of CTC cells often necessitates the extraction of a few hundred CTCs from a sample that includes nearly tens of millions of WBCs, and hundreds of millions of RBCS. As a result, detection and quantification of CTCs is often difficult using smaller sample volumes.

Sample processing that includes centrifugation (and other subtractive techniques) can often cause additional complications in the detection and collection of CTCs and other rare cells in whole blood, because CTCs may inadvertently remain in a bottom region of plasma that in contact with other cellular components of a centrifuged sample volume. For example, CTCs may be lost while aspirating the plasma, lowering the overall capture efficiency of CTCs after centrifugation is complete. This is generally not the case, however, for other types of cells that have higher concentrations in bodily fluids. Accordingly, high-yield consistent extraction of CTCs and other rare cells from smaller sample volumes of whole blood is often difficult to accomplish when utilizing subtractive techniques to separate cellular components.

### SUMMARY

In some implementations of the new additive fluid sample processing methods described herein, techniques that substitute subtractive sample processing with alternative means can be incorporated into magnetic labeling and separation of target entities, such as cells, e.g., rare cells, to improve the capture efficiency of the target entities, e.g., CTCs and other cells, from a small sample volume without significantly impacting targeting efficiency. In one example, a fresh sample volume including cells and/or rare cells can be combined with a diluent to reduce the viscosity of the sample prior to the introduction of conjugated magnetic beads. In this example, the viscosity reduction of the sample volume can be used to decrease non-specific binding of the conjugated magnetic beadings without requiring a centrifugation step. In another example, the conjugated magnetic beads may be initially introduced followed by combination with the diluent. In this example, the reduction of the viscosity can be used to reduce non-specific interactions with a detection surface used to capture of the rare cells. In both of these examples of the new additive sample processing methods, a total number of extracted target entities, such as cells, e.g., rare cells, from a sample volume can be increased significantly compared to the use of traditional subtractive techniques since portions of the sample that may include rare cells are not removed during the sample preparation process prior to detection and extraction.

Additional advantages of the additive sample processing techniques described herein include eliminating a need to use additional equipment and reducing the overall time required for cell analysis. For example, traditional centrifugation-based detection protocols often require 90 to 100 minutes to perform sample preparation of a 7.5 mL of a fluid sample (1.5 to 2 mL of which is removed after centrifugation and aspiration) followed by call capture on a fluidic enclosure. In comparison, the additive techniques enable cell detection within 60 to 70 minutes using smaller sample volumes. In addition, because the additive sample processing techniques do not remove any volume of the original fluid sample, detection results can be obtained with a higher level of purity compared to detection results obtained using centrifugation-based detection protocols (i.e. lower level of non-specific binding between antibodies of conjugated magnetic beads and unwanted cells).

In one general aspect, the present disclosure includes additive, direct dilution methods for isolating target entities, e.g., cells such as rare cells, in a fluid sample. The methods can include the following steps carried on in the following order: adding to the fluid sample a volume of a diluent of at least 0.5 times that of the fluid sample to generate a first mixture, where the volume of the diluent is sufficient to obtain a specified viscosity of the first mixture that is lower than a viscosity of the fluid sample; adding to the first mixture a number of binding moiety-conjugated magnetic beads to generate a second mixture, where binding moieties of the binding moiety-conjugated magnetic beads are capable of specifically binding to one or more ligands expressed on the target entities, e.g., rare target entities, and where the number of binding moiety-conjugated magnetic beads added to the first mixture is sufficient to magnetize the target entities; incubating the second mixture for a time that is between at least 5 and 120 minutes and that is sufficient for the binding moiety-conjugated magnetic beads to bind to target entities in the second mixture, where the viscosity of the second mixture is substantially the same as the specified viscosity of the first mixture and where the viscosity of the second mixture is sufficiently low to inhibit non-specific binding of the binding moiety-conjugated magnetic beads to non-target entities in the fluid sample; flowing a portion of the second mixture into a fluidic chamber using a flow rate that is greater than 1.0 mL/minute; and applying a magnetic force to attract the magnetized target entities in the second mixture to an isolation surface within the fluidic chamber, thereby isolating target entities in an additive method without removing any portion of the original fluid sample.

In another general aspect, the present disclosure includes additive, direct incubation methods for isolating target entities, e.g., rare target entities, in a fluid sample. The methods can include the following steps carried out in the following order: adding to the fluid sample a number of binding moiety-conjugated magnetic beads to generate a first mixture, where binding moieties of the binding moiety-conjugated magnetic beads are capable of specifically binding to one or more ligands expressed on the target entities, and where the number of binding moiety-conjugated magnetic beads added to the fluid sample is sufficient to magnetize the target entities; incubating the first mixture for a time that is between at least 5 and 120 minutes and that is sufficient for the binding moiety-conjugated magnetic beads to bind to target entities in the first mixture; adding to the incubated first mixture a volume of a diluent of at least 0.5 times that of the fluid sample to generate a second mixture, where the volume of the diluent is sufficient to obtain a specified viscosity of the second mixture that is lower than a viscosity of the first mixture; flowing a portion of the second mixture into a fluidic chamber using a flow rate that is greater than 1.0 mL/minute; and applying a magnetic force to attract the magnetized target entities in the second mixture to an isolation surface within the fluidic chamber, where the viscosity of the second mixture is sufficiently low to inhibit non-specific interactions of non-target entities in the fluid sample with the isolation surface, thereby isolating target entities in an additive method without removing any portion of the original fluid sample.

Other versions include corresponding systems and apparatuses configured to perform the actions of the methods. One or more implementations of the methods described herein can include the following optional features. For example, in some implementations, the binding moieties are one or more different antibodies, and the ligands are one or more antigens to which the antibodies specifically bind. The target entities can be cells, e.g., T cells, B cells, white blood cells or subsets of white blood cells, or they can be rare cells, such as CTCs or fetal blood cells found in maternal blood. The target entities can also be bacteria, parasites, one-celled organisms, or specific proteins or other compounds and compositions that can be bound by specific binding moieties.

In some implementations, the direct dilution methods and/or the direct incubation methods include flowing a wash solution into the fluidic chamber after injecting the second mixture into the fluidic chamber.

In some embodiments, the direct dilution methods and/or the direct incubation methods include flowing a buffer solution into the fluidic chamber after flowing the wash solution into the fluidic chamber.

In some implementations, the direct dilution methods and/or the direct incubation methods include passivating the detection or isolation surface of the fluidic chamber prior to injecting the second mixture into the fluidic chamber.

In some implementations, the fluid sample includes a blood sample, e.g., a whole blood sample and the target entities are cells other than red blood cells, and the method further includes flowing a red blood cell lysis buffer through the fluidic chamber using a flow rate of at least 1.0 ml/minute to remove red blood cells from the isolation surface.

In some implementations, the red blood cell lysis buffer flows through the fluidic chamber for at time that is between 1 and 10 minutes.

In some embodiments of the methods described herein, the diluent includes a solution of phosphate-buffered saline, and the diluent has a dilution ratio ranging from 1:1 to 1:4 volume of the diluent to volume of the fluid sample.

In some implementations, the diameter of the binding moiety-conjugated magnetic beads ranges from ten nanometers to fifty micrometers.

In some implementations, the binding moiety-conjugated magnetic beads are conjugated to an EpCAM antibody.

As described herein, “rare target entities” refer to target entities, e.g., cells that have a maximal concentration of 1,000 or fewer cells per millimeter of a fluid sample. The target entities can be cells (e.g., circulating tumor cells, fetal red blood cells in maternal cells) that have concentrations that are less than other types of cells in the fluid sample, e.g., whole blood (e.g., red blood cells, white blood cells, platelets). The rare target entities can be magnetized using different techniques, for example, using magnetic beads conjugated with specific binding moieties, such as antibodies, that are specific to antigens expressed on the surfaces of the rare target entities. In some implementations, the target entities are not “rare” as defined herein, and can include T cells, B cells, white blood cells, subsets of white blood cells, bacteria, and other compounds or compositions that are to be isolated, detected, and/or captured from a liquid sample.

As described herein, “additive” techniques or methods refer to liquid or fluid sample processing techniques that do not remove any portion of an original sample prior to performing a cell extraction and detection procedure. Additive techniques do not include techniques such as centrifugation, filtration, or extraction, where the volume of the original sample is reduced prior to analysis. An example of an additive technique is the addition of a diluent to a sample volume to generate a diluted mixture. Another example of an additive technique is the addition of binding moiety-conjugated magnetic beads to a fluid sample.

As used herein, the term “specifically binds” means that a binding moiety, such as an antibody, binds to a corresponding ligand, such as an antigen, to a significantly greater extent than it will bind to any other non-ligands in a fluid sample.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more implementations are set forth in the accompanying drawings and the description below. Other potential features and advantages will become apparent from the description, the drawings, and the claims.

For the purposes of promoting an understanding of the principles of the present disclosure, reference will now be made to the embodiments and implementations illustrated in the drawings, and specific language will be used to describe the same. It will nevertheless be understood that no limitation of the scope of this disclosure is thereby intended.

### BRIEF DESCRIPTION OF THE DRAWINGS

FIG. 1A is a block diagram that illustrates an example of a cell extraction system.

FIG. 1B is a schematic diagram of another example of a cell extraction system.

FIG. 2A is a flow chart that illustrates an example of a direct dilution protocol.

FIG. 2B is a flow chart that illustrates an example of a direct incubation protocol.

In the drawings, like reference numbers represent corresponding parts throughout.

### DETAILED DESCRIPTION

The new additive sample processing methods described herein include techniques that substitute subtractive sample processing steps with alternative means in the magnetic labeling and separation of target antigens such as cells or rare cells to improve the efficiency of isolation, detection, and/or capture of the target antigens from relatively small sample volumes without significantly impacting targeting efficiency. In one example of the so-called “direct dilution” methods, a fresh sample volume including rare cells is added to a diluent to reduce the viscosity of the sample prior to the introduction of conjugated magnetic beads. In this method, the viscosity reduction of the sample volume is used to decrease non-specific binding of the conjugated magnetic beadings without requiring a centrifugation step. In an example of the so-called “direct incubation” methods, conjugated magnetic beads are initially added to and incubated with the sample fluid followed by the addition of a diluent. In this example, the reduction of the viscosity is used to reduce non-specific interactions with an isolation surface used to capture the target entities, such as rare cells. In both of these additive processing methods, a total number of extracted rare cells from a sample volume can be increased compared to the use of traditional subtractive techniques, because portions of the sample that may include rare cells are not removed during the sample preparation process prior to detection and extraction.

As described herein, a “direct dilution method” refers to the use of addtive techniques to isolate and capture rare target entities in a fluid sample without removing portions of the original fluid sample. For example, during a direct-dilution method, a volume of a diluent is initially added to a fluid sample to generate a mixture with a reduced viscosity set to a specified viscosity level. Binding moiety-conjugated magnetic beads are then added to the mixture to magnetically label rare cells of interest. The mixture containing the fluid sample and the conjugated magnetic beads are then incubated for a specified period of time to allow antibodies of the magnetic beads to bind to specific antigens expressed on the surfaces of the rare cells of interest. The mixture can then be flowed into a fluidic chamber, e.g., a microfluidic chamber. A magnetic force is then applied to capture the magnetically labeled rare target entities within the microfluidic chamber, e.g., using a magnet placed underneath the microfluidic chamber.

As described herein, a “direct incubation method” refers to an alternative technique to the direct-dilution protocol where binding moiety-conjugated magnetic beads are added to the fluid sample before adding a diluent to the original fluid sample. The mixture containing the fluid sample and the conjugated magnetic beads are initially incubated for a specified period of time to allow antibodies of the magnetic beads to bind to specific antigens expressed on the surfaces of the rare cells of interest. A volume of a diluent is then added to the mixture to generate a diluted mixture with a reduced viscosity set to a specified viscosity level. The mixture is then injected into a fluidic chamber, e.g., a microfluidic chamber. A magnetic force is then applied to capture the magnetically labeled rare cells within the microfluidic chamber using a magnet placed underneath the microfluidic chamber.

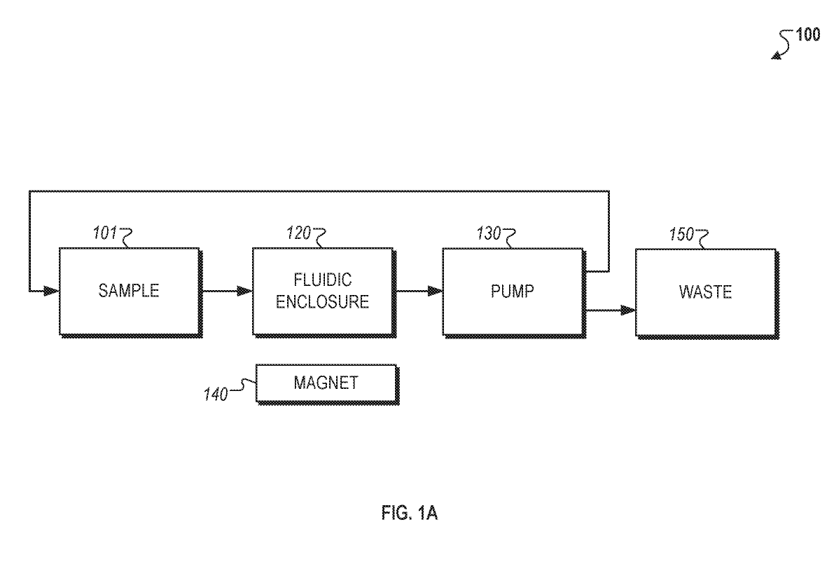

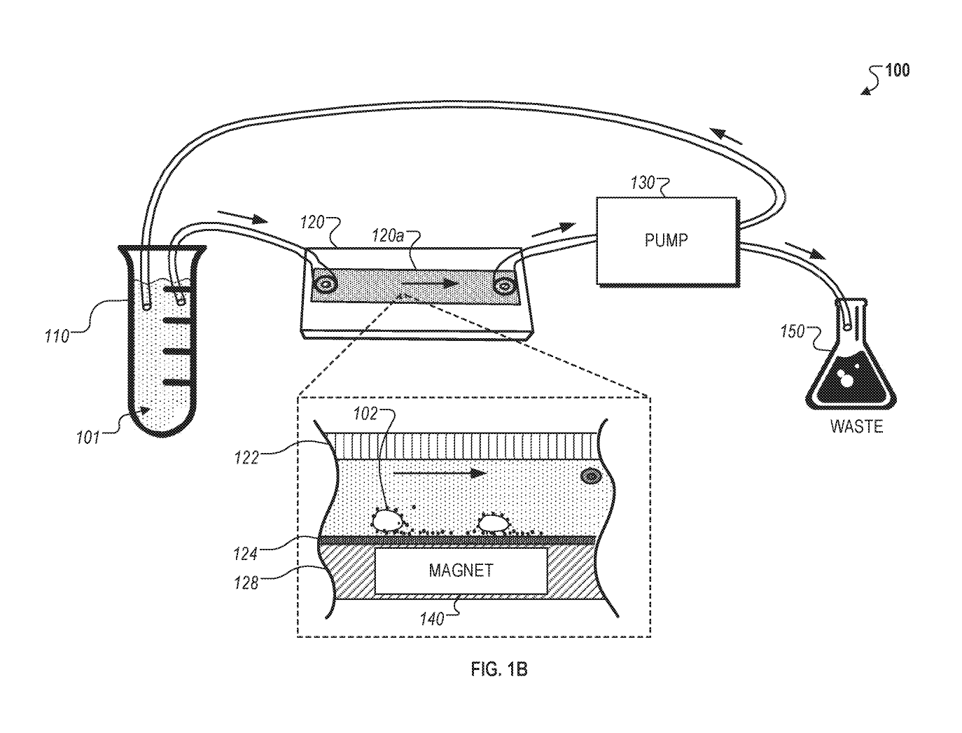

FIG. 1A is a block diagram of a basic cell extraction system 100A. The system 100A includes a sample container 110 that stores a mixture generated using either the direct-dilution or the direct-incubation methods described in more detail below. The mixture includes a fluid sample, a diluent, and magnetic beads that are conjugated with ligand binding moieties such as antibodies. The fluid sample includes rare target entities that are magnetized based on the specific binding of antibodies of the conjugated magnetic beads and antigens that are expressed on the surfaces of the rare target entities. The mixture is flown through a fluidic enclosure 120 to capture the rare target entities that are magnetized with the use of a magnetic force supplied by a magnet 140 to attract the magnetized target entities onto an isolation surface of the fluidic enclosure 120. The fluid within the mixture flows through the fluidic enclosure 120 with the use of a peristaltic pump 130. The mixture that exits the fluidic enclosure 120 can either be disposed of in a waste container 150, or re-circulated back to the sample container 110. In some implementations, the portion of the mixture that is re-circulated back to the sample container 110 can be re-flowed through the fluidic enclosure 120 in order to capture residual target entities that were not previously captured within the fluidic enclosure 120 when the mixture was initially flown through.

FIG. 1B is a schematic diagram that illustrates another example of a cell extraction system 100B. The system 100B includes the sample container 110 that holds a fluid sample 101, the fluidic enclosure 120 including a fluidic chamber 120a, the peristaltic pump 130, the magnet component 140 placed underneath the fluidic enclosure 120, and the waste container 150. The fluidic enclosure 120 can be connected to the peristaltic pump 130 or another device or arrangement for delivery of fluids through a fluidic circuit. A valve system or a plurality of valves can also be used so that the pump 130 can direct the fluid to either to a waste container 150 or back to sample tube 110 for recirculation through the fluidic system. The present methods can also be used in systems such as those described in U.S. patent application Ser. Nos. 12/601,986, 14/001,963, and 14/037,478, the contents of which are all incorporated herein by reference in their entireties.

The fluidic enclosure 120 includes bodies 122 and 128, which define a fluidic channel in which a sample flows from an inlet port connected to the tube 110 to an outlet port connected to the pump 130. The lower body 128 includes an isolation surface 124 that interacts with magnetized rare target entities 102 in the fluid sample 101. The interaction between the magnetized rare target entities 102 and the isolation surface 124 allow for the isolation and capture of rare target entities as described in more detail below.

The lower body 128 of the fluidic enclosure 120 can be a solid surface (e.g., a glass slide, or other materials including silicon, silicon-dioxide, silicon-nitride, glass, PDMS, SU-8 or plastic). The fluidic enclosure 120 can optionally be composed of a PDMS spacer in contact with the bodies 122 and 124, another PDMS/transparent film sheet below the surface 124, and a glass cover slide that accommodates inlet and outlet tubing, and an outer casing that holds the assembly together which may include another glass slide at the bottom. The isolation surface 124 may have a surface area that ranges from 100 μm² to 50 cm² (e.g., 500 μm², 1 cm², 5.0 cm², 10 cm², or 20 cm²) with a minimum effective dimension (width, length, diameter or thickness) of 10 μm.

The magnet component 140 can be used to generate a magnetic field (with magnetic flux densities ranging from 0.01 Telsa to 100 Tesla, e.g., 1.0, 10, 25, 50, 75, or 100 Tesla) within the fluidic enclosure 120 (whose volume can range from 1 mm³ to 10,000 mm³) from within or outside the fluidic enclosure 120 in a manner so as to capture the magnetic beads and entities bound to the magnetic beads (e.g., the magnetized rare target entities 102) by attracting them towards the isolation surface 124 inside the fluidic enclosure 120. This can be accomplished by either inserting/attaching one or multiple permanent or electromagnets to the lower body 128 of the enclosure 120, or by incorporating magnet patterns made of magnetic, paramagnetic, or superparamagnetic materials and electronic circuits to generate magnetic fields.

Rare target entities 102 (or “rare cells”) can include CTCs within the fluid sample 101, and can be isolated and detected based on using binding moiety-conjugated magnetic beads to magnetically label the rare target entities 102 using antibody-antigen binding. For instance, the rare target entities 102 can be bound to magnetic beads that are functionalized with antibodies that recognize specific surface antigens. Once the rare target entities 102 have been magnetically labeled, the fluid sample 101 can be injected into the fluidic enclosure 120 and flown through the fluidic chamber 120a that accommodates the isolation surface 124. The rare target entities 102 that are attached to the magnetic particles can then be brought to the isolation surface 128 by means of a magnetic force provided by the magnet 140 as described above. An exemplary system is disclosed in U.S. application Ser. No. 12/601,986 (U.S. Pub. App. 2010/0330702 to Savran et al.). In some implementation, the target entities 102 are not “rare” as defined herein, and can include, for example, T cells, B cells, white blood cells, or subsets of white blood cells

Prior to the introduction of the fluid sample 101, the chamber 120a is initially filled with a buffer, e.g., 1% bovine serum albumin (BSA) in phosphate-buffered saline (PBS) solution (10 mg/mL), and incubated at room temperature (RT) or 4° C. for over at least about 5 minutes, e.g., 15, 30, 45, or 60 minutes, up to 90 or 120 minutes or more, to passivate the chamber 120a and the accompanying isolation surface 124 for reducing non-specific binding from cells, beads and other entities that are not the rare target entities 102. In some instances, in addition to PBS solution, other buffer solutions such as tris-buffered saline (TBS) can also be used. The concentration of BSA can range from 0 to 10% (100 mg/mL) or more narrowly from 0.1% (1 mg/mL) to 5% (50 mg/mL). The passivated chamber 120a is washed with a buffer solution prior to the introduction of the fluid sample 101 to remove excess BSA. In one implementation, surface blocking can be achieved with agents other than BSA such as polyethylene glycol (PEG), polyvinyl alcohol, polyvinylpyrrolidone, polyacrylic acid, polyacrylic maleic acid, hexadecanoic acid, or various forms of zwitteronic materials. Alternatively, detergents such as Tween (specifically Tween-20) and Triton (specifically Triton X-100) can also be used to block the surface and help reduce nonspecific binding.

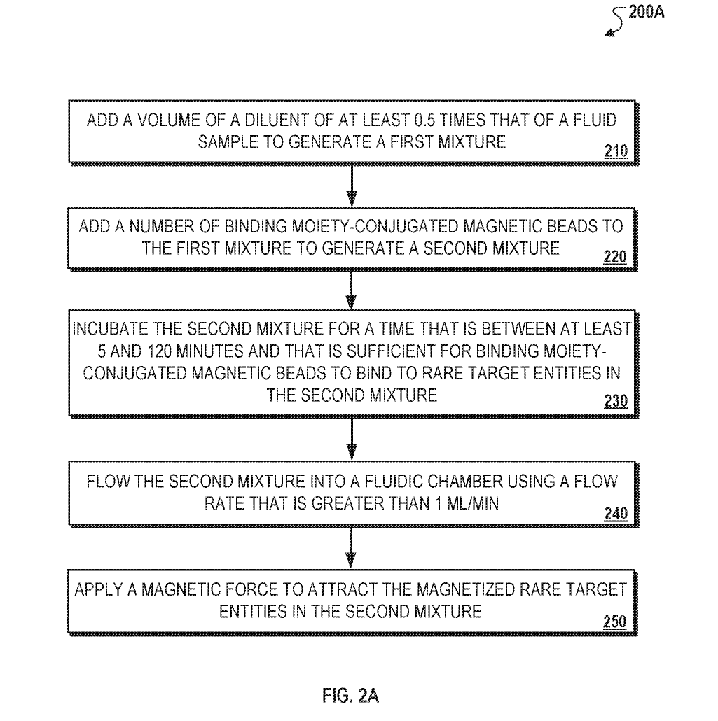

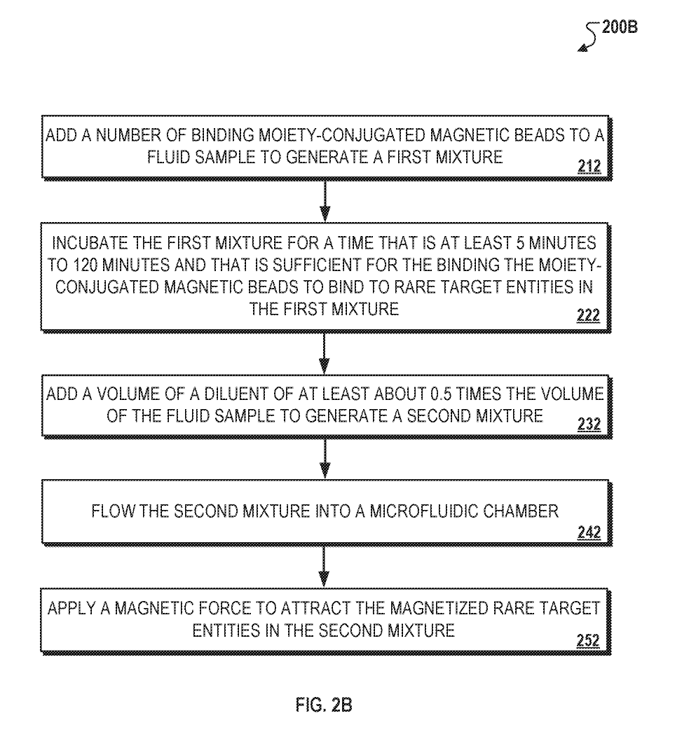

FIG. 2A is a flow chart that illustrates an example of a direct-dilution method 200A for isolating rare isolating rare target entities in a fluid sample. Briefly, the method 200A includes adding a volume of diluent at least 0.5 times that of a fluid sample to generate a first mixture (210), adding a number of binding moiety-conjugated magnetic beads to the first mixture to generate second mixture (220), incubating the second mixture for a time that is between at least 5 and 120 minutes and that is sufficient for the binding moiety-conjugated magnetic beads to bind rare target entities in the second mixture (230), flowing a portion of the second mixture into a microfluidic chamber using a flow rate that is greater than 1.0 mL/minute (240), and applying a magnetic force to attract the magnetized rare target entities in the second mixture (250).

As described above, the direct-dilution method 200A refers to an additive sample processing technique where the fluid sample 101 is initially diluted prior to incubating the fluid sample 101 with antibody-conjugated magnetic beads. The fluid sample 101 includes rare target entities 102, which include CTCs as an example. The direct-dilution method 200A can be performed to remove to need to perform centrifugation in order to process whole blood to enable specific binding between antibodies conjugated to the magnetic beads and target antigens expressed on the surfaces of the rare target entities 102.

In more detail, the method 200A includes adding a volume of diluent at least 0.5 times that of a fluid sample to generate a first mixture (210). The fluid sample 101 (e.g., whole blood obtained from a subject, e.g., a cancer patient containing CTCs; or from a healthy donor and then spiked with cultured cell lines), is initially diluted with PBS solution with a 1:1 dilution ratio to generate a first fluid mixture. In some instances, the dilution ratio can range from 1:0.1 to 1:10 (fluid: PBS) or more narrowly from 1:0.5 to 1:4 (fluid:PBS). In some implementations, PBS can be replaced with, or combined with, other buffers or solutions as well as RBC lysis buffer solution can alternatively be used to dilute the fluid sample 101.

The dilution reduces the viscosity and the overall density of the fluid mixture relative to whole blood such that, if the fluid mixture is exposed to a solid surface, i.e. the isolation surface 124 within the fluidic enclosure 120, a number of entities that are in immediate vicinity of the isolation surface 124 is reduced. As a result, the probability of particulate matter such as cells and molecules within the diluted mixture encountering each other is lowered. As a result, the non-specific binding of entities, as well as the fluidic drag force of the fluid mixture as it flows through the fluidic chamber 120a, are also reduced.

In some implementations, the fluid sample 101 may be a fluid that is different from whole blood. For example, other types of bodily fluids that contain cell such as ascites, pleural fluids, mucus, saliva, or urine may be analyzed instead of blood. In such implementations, even though the dilution also increases the total volume that needs to be processed, the fluidic enclosure 120 can use techniques to provide high volumetric throughput to accommodate such large sample volumes (1 mL to 1 Liter).

The method 200A includes adding a number of binding moiety-conjugated magnetic beads to the first mixture to generate second mixture (220). The binding moieties can be antibodies for target antigens overexpressed on the rare target entities 102 (e.g., epithelial cell adhesion molecule, EpCAM, and epidermal growth factor receptor, EGFR), and they are initially conjugated to magnetic beads. The diameter of the magnetic beads used can vary from 10 nm to 50 μm or more narrowly from 100 nm to 5 μm. The antibodies can be conjugated with the magnetic beads through biotin and streptavidin interaction, but they can also be bound through other covalent interactions such as amine-based conjugation or non-covalent interactions. Other standard conjugation techniques can also be used. The antibody-conjugated magnetic beads are then added to the diluted fluid sample generated in step 210.

In one implementation, 20 μL streptavidin conjugated superparamagnetic beads (10 mg/mL) are saturated with excess amounts of biotinylated antibodies (10 μL, 0.2 mg/mL) in PBS solution and incubated at room temperature (RT: 20° C. to 25° C.) for 1 hour, followed by rinsing with PBS solution 3 times on a magnetic stand and re-suspending in PBS. Depending on the number of magnetic beads used and the binding capacity of the magnetic beads, as well as the fluid sample 101 analyzed, the volumetric ratio of the streptavidin magnetic beads (10 mg/mL) to the biotinylated antibody (0.2 mg/mL) can range from 10:1 to 1:10, and the incubation period can range from 5 minutes to 2 hours. During the incubation, a fluid containing the antibody-conjugated magnetic beads and the diluted fluid sample can be placed on a device that enhances the mixing through rocking, rotating, shaking, or agitating mixture, or a combination of some or all of these techniques.

The method 200A includes incubating the second mixture for a time that is between at least 5 and 120 minutes and that is sufficient for the binding moiety-conjugated magnetic beads to bind rare target entities in the second mixture (230). After dilution, the fluid mixture containing antibody-conjugated magnetic beads and the diluted fluid sample are incubated at room temperature between 5 minutes to 5 hours, or more typically from 15 minutes to an hour. The magnetic beads conjugated with anti-EpCAM (anti-EpCAM beads) are the most common antibody-beads (ab-beads) used, although different antibodies can also be used including antibodies against the epidermal growth factor (EGFR), the carcinoembryonic antigen (CEA), prostate specific membrane antigen (PSmA), folate receptor (FR), prostate specific antigen (PSA), and vimentin. The antibody-conjugated magnetic beads can also be incubated with the diluted fluid sample mixture, along or in combination with, magnetic beads conjugated with other kinds of antibodies (e.g., a cocktail of ab-beads). The total amount of ab-beads used depends on the total volume of the sample mixture, which can range from 0.1 μL (1 μg) to 10 μL (100 μg) per mL of the diluted blood, or more narrowly from 1 μL (10 μg) to 4 μL (40 μg) per mL of the diluted blood. During the incubation, the fluid mixture can be placed on a device that enhances the mixing through rocking, rotating, shaking, or agitating the sample 101, or a combination of some or all of them. In one implementation, antibodies may be replaced with other molecules such as aptamers, peptides, proteins, small molecules, DNA or RNA.

The method 200A includes flowing a portion of the second mixture into a microfluidic chamber using a flow rate that is greater than 1.0 mL/minute (240). After incubation, the mixture containing the ab-beads and the diluted fluid mixture is then introduced into the fluidic enclosure 120 to enable the detection of rare target entity 102 (e.g. CTCs). The mixture is injected into the fluidic chamber 120a and flowed from the inlet of the fluidic chamber 120a to the outlet of the fluidic chamber 120a at a certain flow rate.

The method 200A includes applying a magnetic force to attract the magnetized rare target entities in the second mixture (250). The magnet component 140 is generally situated underneath the fluidic enclosure 120, or underneath chamber 120a within the external housing of the fluid enclosure 120. The magnet component 140 is calibrated to exert a magnetic force sufficient to pull the magnetized rare target entities 102 towards the isolation surface 124 and to retain the magnetized target entities 102 at a location on the surface 124 as fluid flows through the microfluidic chamber 120 from the inlet port to the outlet port (e.g., during wash steps). As an example, the magnet 130 is an NdFeB Cube Magnet (about 5×5×5 mm) with a measured surface flux density and gradient of 0.4 T and 100 T/m, respectively. In other examples, other magnets including, but not limited to, larger or smaller permanent magnets made of various materials, and electromagnets that are commercially available or manufactured using standard or microfabrication procedures and that are capable of generating time-varying magnetic fields, can also be used.

At the end of the cell capture process, the chamber 120a is washed with 1 to 10 mL of PBS solution (or more narrowly with 2 to 5 mL of PBS solution) at the operational flow rate, following by introducing RBC lysis buffer and incubation for up to 5 minutes to remove RBCs left in the chamber 120a. In one implementation, the RBC lysis buffer is circulated through the fluidic enclosure 120 using a flow rate between 0.01 to 20 mL/min. The chamber 120a is then washed with 1 to 10 mL (or more narrowly with 2 mL) PBS solution and subjected to immunofluorescence analysis.

In some implementations, a portion of the fluid mixture exiting the fluidic chamber 120 bypasses the waste container 150 and is re-circulated back into the sample container 110, e.g., using the peristaltic pump 130, gravity, or some other pump. In certain implementations, the optimal flow rate can be 2 mL/min. However, the operational flow rate can range from 0.01 to 20 mL/minute, e.g., 0.05, 0.1, 1.0, 2.5, 5.0, 7.5, 10.0, 12.5, 15.0, 17.5, or 20.0 mL/minute. The circulation time is dependent upon the total volume of the sample mixture and can range from 5 seconds to up to 15 minutes, e.g., 10, 20, or 60 seconds, or 2, 5, 7, 10, 12, or 15 minutes. As a result, the mixture flowing through the fluidic chamber 120a can be re-circulated multiple times over to capture any residual target entities 102 that were not initially captured through prior circulations. Alternatively, the mixture can also be passed through the fluidic enclosure once without any recirculation.

In one implementation, magnetized rare target entities 102 that are captured on the isolation surface can analyzed. The magnetized rare target entities 102 are first fixed using a 4% paraformaldehyde (PFA) solution in PBS for 10 to 15 minutes, and then permeabilized using a 0.1 to 0.2% Triton X-100 solution in PBS for 10 minutes while the microchip is in the fluidic chamber 120a. Antibodies conjugated with fluorescent dyes are subsequently introduced to label the magnetized target entities 102 that have been captured on the isolation surface 124. In one implementation, anti-cytokeratin monoclonal antibodies conjugated with FITC (anti-CK-FITC), anti-CD45 monoclonal antibodies (to rule out WBCs) conjugated with phycoerythrin (anti-CD45-PE), and 4,6-diamidino-2-phenylindole (DAPI) to verify nucleated cells are introduced into the chamber 120a at the same time and incubated for 15 min at room temperature to label the cells. To maintain the viability of the magnetized target entities 102 captured, the fixation and permeabilization steps prior to fluorescent staining can be optionally performed. However, the fluorescent staining time will generally need to be extended to up to 30 minutes if no fixation is used.

The magnetized target entities 102 captured on the isolation surface 124 can be then subjected to fluorescent microscopy while still in the chamber 120a for identification and enumeration. If the magnetized target entities 102 are tumor cells, they are identified based on a combination of factors including the size (10-30 μm) and shape (close to circular) of the cells, and the fluorescent emissions (CK+, DAPI+ and CD45−). The entities that do not fit this description may have non-specifically bound to either the beads and/or the chip surface and therefore are not scored as a tumor cell. Other techniques can be used to stain or recognize other markers within or on the surface of the cells, which may not involve the use of fluorescence.

FIG. 2B is a flow chart that illustrates an example of a direction incubation method 200B for isolating rare isolating rare target entities in a fluid sample. Briefly, the method 200B includes adding a number of binding moiety-conjugated magnetic beads to a fluid sample to generate first mixture (212), incubating the first mixture for a time that is at least 5 minutes to 120 minutes and that is sufficient for binding the moiety-conjugated magnetic beads to bind to rare target entities in the first mixture (222), adding a volume of a diluent of at least about 0.5 times the volume of the fluid sample to the incubated first mixture to generate a second mixture (232), flowing a portion of the second mixture into a microfluidic chamber (242), and applying a magnetic force to attract the magnetized rare target entities in the second mixture (252).

As described above, the direct incubation method 200B refers to an additive sample processing technique where the fluid sample 101 is diluted after incubating the fluid sample 101 with antibody-conjugated magnetic beads. Similar to the direct dilution method 200A, the direct incubation method 200B can be performed to remove to need to perform centrifugation in order to process whole blood to enable specific binding between antibodies conjugated to the magnetic beads and target antigens expressed on the surfaces of the rare target entities 102.

In more detail, the method 200B includes adding a number of binding moiety-conjugated magnetic beads to a fluid sample to generate first mixture (212). Ab-beads are initially introduced into the fluid sample 101 to generate a mixture in which antibodies conjugated to the magnetic beads specifically bind to antigens that are expressed on the surfaces of the rare target entities 102. For example, ab-beads are directly added into the fluid sample 101 in a manner similar to the techniques described above with respect to step 220. The total amount of ab-beads used can range from 0.1 μL (1 μg) to 10 μL (100 μg) per mL of the blood or more narrowly from 0.5 μL (5 μg) to 4 μL (40 μg) per mL of the blood.

The method 200B includes incubating the first mixture for a time that is at least 5 minutes to 120 minutes and that is sufficient for binding the moiety-conjugated magnetic beads to bind to rare target entities in the first mixture (222). The mixture containing the ab-beads and the fluid sample 101 can be incubated between 5 minutes to 2 hours depending on the sample volume analyzed and the amount of ab-beads used in a manner similar to the techniques described above with respect to step 230.

The method 200B includes adding a volume of a diluent of at least about 0.5 times the volume of the fluid sample to the incubated first mixture to generate a second mixture (232). The incubated mixture containing ab-beads and the fluid sample 101 is diluted with buffer solution such as PBS solution at a ratio of 1:1 in a manner similar to the techniques described above with respect to step 210. The dilution ratio can range from 1:0.1 to 1:10 (mixture:PBS) or more narrowly from 1:0.5 to 1:4 (mixture:PBS).

The method 200B includes flowing a portion of the second mixture into a microfluidic chamber using a flow rate that is greater than 1.0 mL/minute (242). The diluted mixture containing the ab-beads, the fluid sample 101, and the diluent is injected into the microfluidic chamber 120a in a manner similar to the techniques described above with respect to step 240.

The method 200B includes applying a magnetic force to attract the magnetized rare target entities in the second mixture (252). The magnet 140 can be used to exert a magnetic force sufficient to attract the magnetized rare target entities 102 within the diluted mixture to the isolation surface 124 in a manner similar to the techniques described above with respect to step 250.

### Examples

The following examples do not limit the new additive processing methods described herein.

An experiment was conducted to evaluate the capture efficiency of rare target entities using the direct-dilution method 200A and the direction dilution method 200B described above. Previously identified cancer cell lines were initially spiked into healthy human blood as described above. In one exemplary process, a known number (e.g., between 25 to 85 cells) of MCF-7 cells (breast cancer cell line) were first spiked in 1 mL of healthy blood and diluted to 2 mL with PBS solution, 4 μL (40 μg) of anti-EpCAM beads were then added into the diluted sample and incubated at RT for at least 75 minutes. The sample mixtures were subsequently circulated in the fluidic enclosure 120 at a flow rate of 2 mL/min for 2 minutes while a magnet was placed under the fluidic enclosure 120 to draw magnetic particles as well as magnetic particle-bound cells to a solid surface placed inside the fluidic enclosure 120, following by washed with 3 mL of PBS solution. Next, RBC (red blood cell) lysis buffer was introduced into the fluidic enclosure 120 and left for a 5-minute incubation and again the chamber 120a washed with 2 mL of PBS solution. The cells captured on the microchip were then fixed, permeabilized and fluorescently stained according to the protocol described in the previous section. The detected cells were then identified and counted under a fluorescent microscope.

Results from the experiment conduct illustrate that both the direct dilution method 200A and the direct-incubation method 200B can enable higher detection yields of rare target entities on a consistent basis compared to traditional sample processing techniques involving centrifugation. This is because the centrifugation and subsequent aspiration steps, which often vary between samples and users performing the aspirations, are not necessary to prepare a fluid sample for cell detection and analysis.

Additional advantages of the additive sample processing techniques described throughout include eliminating a need to use additional equipment and reducing the overall time required for cell analysis. For example, traditional centrifugation-based detection protocols often require 90 to 100 minutes to perform sample preparation of a 7.5 mL of a fluid sample (1.5 to 2 mL of which is removed after centrifugation and aspiration) followed by call capture on a fluidic enclosure. In comparison, the additive techniques enable cell detection within 60 to 70 minutes using smaller sample volumes. In addition, because the additive sample processing techniques do not remove any volume of the original fluid sample, detection results can be obtained with a higher level of purity compared to detection results obtained using centrifugation-based detection protocols (i.e. lower level of non-specific binding between antibodies of conjugated magnetic beads and unwanted cells).

For example, an experiment conducted to compare the capture efficiencies between additive sample processing techniques and centrifugation-based detection techniques. Results showed that use of the centrifugation-based techniques led to a total number of 800 to 19,900 non-target cells (with an estimated average of 4,000 cells) being captured on a fluidic enclosure with a 7.5 mL of whole blood. In comparison, use of the additive techniques led to a ten-fold increase in purity with only 10 to 1,500 non-target cells (with an estimated average of 400 cells) being captured on a fluidic enclosure with same volume of whole blood.

### Other Embodiments

A number of embodiments and implementations have been described. Nevertheless, it will be understood that various modifications can be made without departing from the spirit and scope of the invention. In addition, other steps can be provided, or steps can be eliminated, from the described methods, and other components can be added to, or removed from, the described systems. Accordingly, other implementations are within the scope of the following claims.

1. An additive, direct dilution method for isolating target entities in a fluid sample, the method comprising the following steps carried out in the following order:
adding to the fluid sample a volume of a diluent of at least 0.5 times that of the fluid sample to generate a first mixture, wherein the volume of the diluent is sufficient to obtain a specified viscosity of the first mixture that is lower than a viscosity of the fluid sample;
adding to the first mixture a number of binding moiety-conjugated magnetic beads to generate a second mixture, wherein binding moieties of the binding moiety-conjugated magnetic beads are capable of specifically binding to one or more ligands expressed on the target entities, and wherein the number of binding moiety-conjugated magnetic beads added to the first mixture is sufficient to magnetize the target entities;
incubating the second mixture for a time that is between at least 5 and 120 minutes and that is sufficient for the binding moiety-conjugated magnetic beads to bind to target entities in the second mixture, wherein the viscosity of the second mixture is substantially the same as the specified viscosity of the first mixture and wherein the viscosity of the second mixture is sufficiently low to inhibit non-specific binding of the binding moiety-conjugated magnetic beads to non-target entities in the fluid sample;
flowing a portion of the second mixture into a fluidic chamber at a flow rate that is greater than 1.0 mL/minute; and
applying a magnetic force to attract the magnetized rare target entities in the second mixture to an isolation surface within the fluidic chamber, thereby isolating rare target entities in an additive method without removing any portion of the original fluid sample.

2. The method of claim 1, wherein the target entities are rare cells.

3. The method of claim 1, wherein the diluent comprises a buffer solution.

4. The method of claim 1, wherein the binding moieties are one or more different antibodies, and the ligands are one or more antigens to which the antibodies specifically bind.

5. The method of claim 1, further comprising flowing a wash solution into the fluidic chamber after flowing the second mixture into the fluidic chamber.

6. The method of claim 1, further comprising flowing a buffer solution into the fluidic chamber after injecting the wash solution into the fluidic chamber.

7. The method of claim 1, further comprising passivating the detection surface of the fluidic chamber prior to flowing the second mixture into the fluidic chamber.

8. The method of claim 1, wherein the fluid sample comprises blood and the method further comprises flowing a red blood cell lysis buffer through the fluidic chamber using a flow rate of at least 1.0 ml/minute to remove red blood cells from the isolation surface.

9. The method of claim 1, wherein the red blood cell lysis buffer flows through the fluidic chamber for at time that is between 1 and 10 minutes.

10. The method of claim 1, wherein the diluent comprises a solution of phosphate-buffered saline, and wherein the diluent has a dilution ratio ranging from 1:1 to 1:4 volume of the diluent to volume of the fluid sample.

11. The method of claim 1, wherein a diameter of the binding moiety-conjugated magnetic beads ranges from ten nanometers to fifty micrometers.

12. (canceled)

13. The method of claim 1, wherein flowing the second mixture into the fluidic chamber comprises:
redirecting at least a portion of the second mixture that exits the fluidic chamber to a container that holds or a conduit that conveys the portion of the second mixture; and
flowing the portion of the second mixture from the container or through the conduit into an inlet of the fluidic chamber.

14. An additive, direct incubation method for isolating target entities in a fluid sample, the method comprising the following steps carried out in the following order:
adding to the fluid sample a number of binding moiety-conjugated magnetic beads to generate a first mixture, wherein binding moieties of the binding moiety-conjugated magnetic beads are capable of specifically binding to one or more ligands expressed on the target entities, and wherein the number of binding moiety-conjugated magnetic beads added to the fluid sample is sufficient to magnetize the target entities;
incubating the first mixture for a time that is between at least 5 and 120 minutes and that is sufficient for the binding moiety-conjugated magnetic beads to bind to target entities in the first mixture;
adding to the incubated first mixture a volume of a diluent of at least 0.5 times that of the fluid sample to generate a second mixture, wherein the volume of the diluent is sufficient to obtain a specified viscosity of the second mixture that is lower than a viscosity of the first mixture;
flowing a portion of the second mixture into a fluidic chamber using a flow rate that is greater than 1.0 mL/minute; and
applying a magnetic force to attract the magnetized rare target entities in the second mixture to an isolation surface within the fluidic chamber, wherein the viscosity of the second mixture is sufficiently low to inhibit non-specific interactions of non-target entities in the fluid sample with the isolation surface, thereby isolating target entities in an additive method without removing any portion of the original fluid sample.

15. The method of claim 14, wherein the binding moieties are one or more different antibodies, and the ligands are one or more antigens to which the antibodies specifically bind.

16. The method of claim 14, further comprising flowing a wash solution into the fluidic chamber after flowing the second mixture into the fluidic chamber.

17. The method of claim 14, further comprising flowing a buffer solution into the fluidic chamber after flowing the wash solution into the fluidic chamber.

18. The method of claim 14, further comprising passivating the detection surface of the fluidic chamber prior to flowing the second mixture into the fluidic chamber.

19. The method of claim 14, wherein the fluid sample comprises blood and the method further comprises flowing a lysing solution into the fluidic chamber after flowing the second mixture into a fluidic chamber, wherein the lysing solution lyses erythrocytes in the second mixture that are in contact with the detection surface of the fluidic chamber.

20. The method of claim 14, wherein the diluent comprises a solution of phosphate-buffered saline, and wherein the diluent has a dilution ratio ranging from 1:1 to 1:4 volume of the diluent to volume of the fluid sample.

21. The method of claim 14, wherein flowing the second mixture into the fluidic chamber comprises:
redirecting at least a portion of the second mixture that exits the fluidic chamber to a container that holds or a conduit that conveys the portion of the second mixture; and
flowing the portion of the second mixture from the container or through the conduit into an inlet of the fluidic chamber.

22-23. (canceled)
